(19) United States
(12) Patent Application Publication
(10) Pub. No.: US 2021/0278653 A1
(43) Pub. Date: Sep. 9, 2021

(54) CONTROL DEVICE AND MEDICAL OBSERVATION SYSTEM

(71) Applicant: Sony Olympus Medical Solutions Inc., Tokyo (JP)

(72) Inventor: Kazuhiro YAMAGUCHI, Tokyo (JP)

(73) Assignee: Sony Olympus Medical Solutions Inc., Tokyo (JP)

(21) Appl. No.: 17/149,746

(22) Filed: Jan. 15, 2021

(30) Foreign Application Priority Data
Mar. 4, 2020 (JP) 2020-037231

(51) Int. Cl. G02B 21/36; G02B 21/00; A61B 90/00

(52) U.S. Cl. CPC G02B 21/365; G02B 21/0012; A61B 2562/0219; A61B 2090/0807; A61B 90/08

(57) ABSTRACT

A control device includes: an acquiring unit configured to acquire a detection value from a sensor that detects a posture of a head of a user who operates an imaging device that images an observation target; and a control unit configured to generate an operation instruction signal for the imaging device using a reference value set by the user viewing initialization information displayed by a display device and the detection value.

### CROSS-REFERENCE TO RELATED APPLICATION

This application claims priority from Japanese Application No. 2020-037231, filed on Mar. 4, 2020, the contents of which are incorporated by reference herein in its entirety.

### BACKGROUND

The present disclosure relates to a control device and a medical observation system.

In a medical imaging device that images a very small part of a body to be observed, there has been known a technique for converting a visual field with a gesture (see, for example, Japanese Patent Application Laid-open Publication No. 11-104064). In this technique, a cursor on a screen is moved by a gesture of the head of a surgeon and visual field movement is performed by pressing a separately provided switch to place the position of the cursor in the screen center.

### SUMMARY

Japanese Patent Application Laid-open Publication No. 11-104064 described above adopts a process for converting a movement amount of the head into a movement amount of the cursor and, thereafter, deciding the movement amount of the cursor with the separate switch. Therefore, operation is complicated. Accordingly, there has been a demand for a technique that is able to operate an imaging device with simple operation.

According to one aspect of the present disclosure, there is provided a control device including: an acquiring unit configured to acquire a detection value from a sensor that detects a posture of a head of a user who operates an imaging device that images an observation target; and a control unit configured to generate an operation instruction signal for the imaging device using a reference value set by the user viewing initialization information displayed by a display device and the detection value.

### BRIEF DESCRIPTION OF THE DRAWINGS

FIG. 1 is a diagram schematically illustrating a medical observation system according to a first embodiment;

FIG. 2 is a block diagram illustrating a functional configuration of the medical observation system according to the first embodiment;

FIG. 3 is a diagram (No. 1) schematically illustrating a posture of a head at the time when a user inputs an operation instruction signal;

FIG. 4 is a diagram (No. 2) schematically illustrating the posture of the head at the time when the user inputs the operation instruction signal;

FIG. 5 is a diagram schematically illustrating an example in which a difference between a reference value of a sensor and a detection value is associated with the operation instruction signal;

FIG. 6 is a diagram (No. 3) schematically illustrating the posture of the head at the time when the user inputs the operation instruction signal;

FIG. 7 is a block diagram illustrating a functional configuration of a medical observation system according to a second embodiment;

FIG. 8 is a block diagram illustrating a functional configuration of a medical observation system according to a third embodiment;

FIG. 9 is a timing chart (No. 1) illustrating a relation between an input state of a footswitch and the operation of a microscope device;

FIG. 10 is a timing chart (No. 2) illustrating the relation between the input state of the footswitch and the operation of the microscope device;

FIG. 11 is a timing chart (No. 3) illustrating the relation between the input state of the footswitch and the operation of the microscope device; and

FIG. 12 is a block diagram illustrating a functional configuration of a medical observation system according to a fourth embodiment.

### DETAILED DESCRIPTION

Modes for carrying out the present disclosure (hereinafter referred to as “embodiments”) are explained below with reference to the accompanying drawings.

### First Embodiment

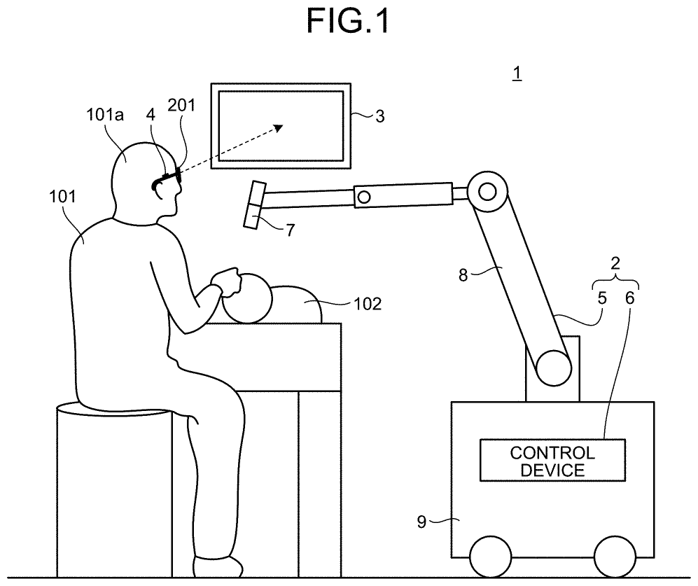

FIG. 1 is a diagram schematically illustrating a medical observation system according to a first embodiment. FIG. 1 illustrates a situation in which a user 101 such as a doctor, who performs a surgical operation using a medical observation system 1, is performing a surgical operation on a patient 102. The medical observation system 1 illustrated in FIG. 1 includes a medical observation apparatus 2, a display device 3, and a sensor 4.

The medical observation apparatus 2 includes a microscope device 5 and a control device 6. The microscope device 5 has a function of an imaging device that images a very small part of a body to be observed and acquires an image signal. The control device 6 has a function of a medical image processing device that performs image processing on the image signal captured by the microscope device 5. The medical observation apparatus 2 according to the first embodiment is a surgical microscope.

The display device 3 is communicably connected to the control device 6 by radio or wire. The display device 3 receives a three-dimensional image signal or a two-dimensional image signal from the control device 6 and displays a three-dimensional image (3D image) based on the three-dimensional image signal or a two-dimensional image (2D image) based on the two-dimensional image signal. The display device 3 includes a display panel made of liquid crystal or organic EL (Electro Luminescence)

The sensor 4 includes a three-axis acceleration sensor. The sensor 4 detects a posture of a head 101a of the user 101 and outputs a detection result to the control device 6. Communication connection between the sensor 4 and the control device 6 may be either wireless or wired. The sensor 4 is attached to eyeglasses 201 worn by the user 101 in a surgical operation. The eyeglasses 201 is one of an active shutter type (a frame sequential type) and a passive type (a circularly polarized light filter type) and is preferably the passive type. In FIG. 1, the sensor 4 is attached to a portion of a temple of the eyeglasses 201. However, a place where the sensor 4 is attached is not limited to this. The sensor 4 may be attached to, for example, a middle part of left and right lenses of the eyeglasses 201. The sensor 4 only has to be able to detect movement of the head 101a of the user 101 and does not have to be attached to the eyeglasses 201. The user 101 does not have to be the doctor and may be, for example, a surgical assistant.

An exterior configuration of the microscope device 5 is explained. The microscope device 5 includes a microscope unit 7 that enlarges and images a microstructure of a body to be observed, a supporting unit 8 that supports the microscope unit 7, and a base unit 9 that holds the proximal end of the supporting unit 8 and incorporates the control device 6.

The microscope unit 7 includes a tubular section formed in a columnar shape. A cover glass is provided on an aperture surface at the lower end portion of a main body section (not illustrated). The tubular section may be gripped by the user and has size for enabling the user to move while gripping the tubular section when the user changes an imaging visual field of the microscope unit 7. Note that the shape of the tubular section is not limited to the cylindrical shape and may be a polygonal cylindrical shape.

The supporting unit 8 includes a plurality of links in an arm section. The links adjacent to each other are turnably coupled via a joint section. A transmission cable for transmitting various signals between the microscope unit 7 and the control device 6 and a light guide for transmitting illumination light generated by the control device 6 to the microscope unit 7 are inserted through a hollow section formed on the inside of the supporting unit 8.

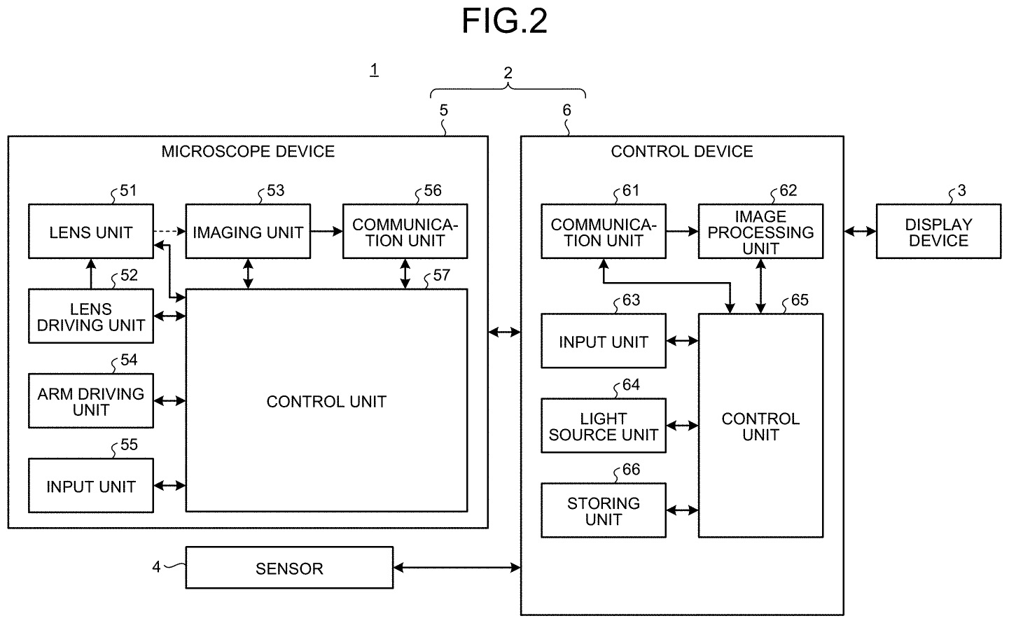

FIG. 2 is a block diagram illustrating a functional configuration of the medical observation apparatus 2. First, a functional configuration of the microscope device 5 is explained. The microscope device 5 includes a lens unit 51, a lens driving unit 52, an imaging unit 53, an arm driving unit 54, an input unit 55, a communication unit 56, and a control unit 57.

The lens unit 51 is configured using a plurality of lenses movable along an optical axis. The lens unit 51 focuses a condensed object image on an imaging surface of an imaging element included in the imaging unit 53. The lens unit 51 includes a focus lens that adjusts a focus and a zoom lens that changes an angle of view. Each of the focus lens and the zoom lens is configured using one or a plurality of lenses. The lens unit 51 includes two position sensors that respectively detect the position of the focus lens and the position of the zoom lens and output the positions to the control unit 57.

The lens driving unit 52 includes actuators that respectively operate the focus lens and the zoom lens under control by the control unit 57 and drivers that drive the actuators under the control by the control unit 57. The imaging unit 53 includes two imaging elements that focus the object image condensed by the lens unit 51 and generate captured images (analog signals) and a signal processing unit that performs signal processing such as noise removal and A/D conversion on image signals (analog signals) from the imaging elements. Visual fields of the two imaging elements have a parallax. The imaging elements are capable of generating a 3D image. The imaging element is configured using an image sensor such as a CCD (Charge Coupled Device) or a CMOS (Complementary Metal Oxide Semiconductor).

The arm driving unit 54 operates each of a plurality of joints included in the supporting unit 8 under the control by the control unit 57. Specifically, the arm driving unit 54 includes actuators provided in joint sections among arms and drivers that drive the actuators.

The input unit 55 receives inputs of an operation signal for the lens unit 51, an operation signal for the arms, and the like. The input unit 55 includes a plurality of switches, a plurality of buttons, and the like provided, on a side surface of the tubular section of the microscope unit 7, in positions where the switches, the buttons, and the like are operable in a state in which the user grips the microscope unit 7.

The communication unit 56 is an interface that performs communication between the communication unit 56 and the control device 6. The communication unit 56 transmits an image signal (a digital signal) generated by the imaging unit 53 to the control device 6 and receives a control signal from the control device 6.

The control unit 57 controls the operation of the microscope device 5 in cooperation with a control unit 65 of the control device 6. The control unit 57 operates the microscope device 5 based on an operation instruction signal, an input of which is received by the input unit 55, and an operation instruction signal sent from the control unit 65 of the control device 6. The control unit 57 receives an operation instruction signal generated by the control unit 65 based on the posture of the head 101a of the user 101 detected by the sensor 4 and operates the arm driving unit 54 based on the operation instruction signal.

The control unit 57 is configured using at least any one processor of a CPU (Central Processing Unit), an FPGA (Field Programmable Gate Array), an ASIC (Application Specific Integrated Circuit), and the like.

A functional configuration of the control device 6 is explained. The control device 6 includes a communication unit 61, an image processing unit 62, an input unit 63, a light source unit 64, the control unit 65, and a storing unit 66.

The communication unit 61 has a function of an acquiring unit that acquires various signals from the sensor 4 and the microscope device 5. The communication unit 61 acquires an image signal captured by the microscope device 5 and transmitted through the transmission cable. The image signal includes information concerning imaging such as a gain adjustment value during the imaging, a focus lens position, a zoom lens position, an exposure time, a diaphragm value. The communication unit 61 has a function of an acquiring unit that acquires information concerning acceleration detected by the sensor 4.

The image processing unit 62 applies various kinds of signal processing to the image signal acquired by the communication unit 61 to thereby generate an image signal for display and outputs the generated image signal to the display device 3. Specifically, the image processing unit 62 performs publicly-known image processing such as wave detection processing, interpolation processing, color correction processing, color emphasis processing, and contour emphasis processing. The image processing unit 62 is configured using at least any one processor of a CPU, an FPGA, an ASIC, and the like.

The input unit 63 receives an input of various kinds of information. The input unit 63 is configured using a user interface such as a keyboard, a mouse, or a touch panel. Note that the input unit 63 may also include a function of at least a part of the input unit 55 of the microscope device 5.

The light source unit 64 generates illumination light to be supplied to the microscope device 5 via the light guide. The light source unit 64 is configured using a solid-state light emitting element such as an LED (Light Emitting Diode) or an LD (Laser Diode), a laser light source, a xenon lamp, a halogen lamp, or the like. The control unit 65 generates, based on a detection value of the sensor 4, an operation instruction signal corresponding to the posture of the head 101a of the user 101 and transmits the operation instruction signal to the control unit 57 of the microscope device 5. At this time, the control unit 65 extracts, using a reference value set in advance as a detection value of the sensor 4 corresponding to a reference posture of the head 101a of the user 101 and a detection value of the sensor 4 at a normal operation time, a difference between the reference posture and a posture at the normal operation time and generates an operation instruction signal based on the difference. Note that the control unit 65 may transmit the detection value of the sensor 4 or a signal corresponding to the detection value to the control unit 57 of the microscope device 5. The control unit 57 may generate the operation instruction signal based on the detection value or the signal corresponding to the detection value received from the control unit 65.

The control unit 65 controls the operation of the control device 6 and collectively controls the operation of the medical observation apparatus 2 in cooperation with the control unit 57 of the microscope device 5. Specifically, for example, the control unit 65 performs wave detection of the image signal acquired by the communication unit 61 and controls light emission of the light source unit 64 and controls an exposure time in the imaging unit 53. The control unit 65 performs control for causing the display device 3 to display the image signal for display generated by the image processing unit 62. The control unit 65 may have a function of changing an angle of view of the image signal for display with electronic zoom. The control unit 65 is configured using at least any one processor of a CPU, an FPGA, an ASIC, or the like. Note that the image processing unit 62 and the control unit 65 may be configured using a common processor.

The storing unit 66 stores various programs for the control device 6 to operate and temporarily stores data on which the control device 6 is performing arithmetic processing. The storing unit 66 is configured using a ROM (Read Only Memory), a RAM (Random Access Memory), or the like.

When the user 101 performs a surgical operation of the head or the like of the patient 102 using the medical observation system 1 having the configuration explained above, the user 101 performs the surgical operation while viewing, via the worn eyeglasses 201, a 3D image displayed by the display device 3.

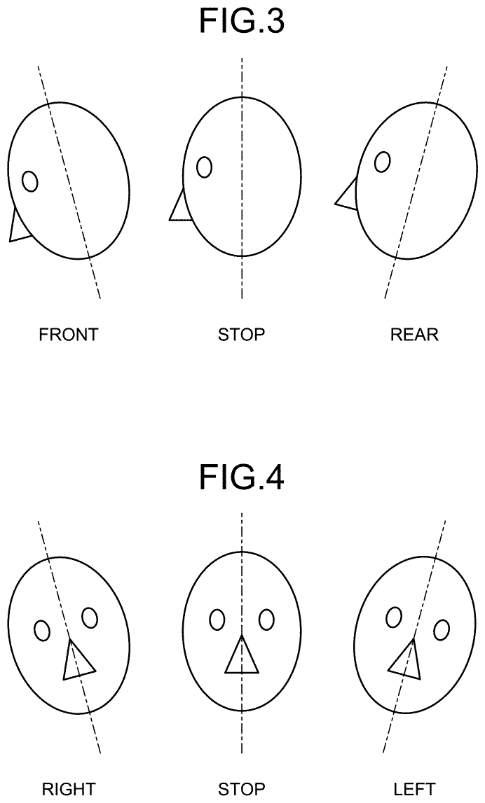

FIGS. 3 and 4 are diagrams schematically illustrating a posture of the head 101a at the time when the user 101 inputs an operation instruction signal in the medical observation system 1. In the first embodiment, the user 101 moves the head 101a to change the posture to thereby input an operation instruction signal for moving an imaging visual field of the microscope device 5. Specifically, as illustrated in FIG. 3, a posture in which the head 101a is tilted forward or rearward corresponds to an instruction for moving the imaging visual field downward or upward. As illustrated in FIG. 4, a posture in which the head 101a is tilted to the left or the right corresponds to an instruction for moving the imaging visual field to the left or the right.

The control unit 65 determines a posture of the head 101a of the user 101 based on a detection result of the sensor 4. At this time, the control unit 65 compares the detection result of the sensor 4 with a reference value set in advance to thereby determine the posture of the head 101a.

First, a setting method for the reference value is explained. When setting the reference value, the control unit 65 displays a predetermined indicator (initialization information) in the center on a screen of the display device 3 and displays, on the screen, a message for urging the user 101 to gaze at the indicator. The indicator has, for example, a cross shape or a circular shape and is not particularly limited if the indicator has a shape that the user 101 may easily gaze at. The control unit 65 may output the message by sound.

Thereafter, the control unit 65 causes the storing unit 66 to store a detection value of a time t, that is, acceleration (Ax(t),Ay(t),Az(t)) at the time t received from the sensor 4 and sequentially calculates, with elapse of time, dispersions of components of the acceleration as statistical values indicating statistical fluctuation in the acceleration in a predetermined period in the time t.

The dispersions in the predetermined period are simply referred to as dispersions. When all of the dispersions of the components are equal to or smaller than a threshold, the control unit 65 calculates averages (Axc, Ayc, Azc) of the components of the acceleration and sets the averages as reference values and causes the storing unit 66 to store the reference values together with information such as a setting date and time. The threshold is a value that is set in order to determine statistical fluctuation. A case in which a dispersion is equal to or smaller than the threshold is equivalent to a case in which a reference of small fluctuation is satisfied. Note that the control unit 65 may cause the storing unit 66 to store identification information of the user 101 in association with the reference value.

When any one of the dispersions of the components of the acceleration is larger than the threshold, the control unit 65 disables the operation instruction signal. At this time, the control unit 65 may disable the operation instruction signal by stopping the generation of the operation instruction signal or may disable the operation instruction signal by stopping the transmission of the generated operation instruction signal to the microscope device 5. The control unit 65 causes the display device 3 to display an error message, display an indicator in the center on the screen of the display device 3, and display a message for urging the user 101 to gaze at the indicator. A color, a shape, and the like of the indicator at this time may be changed from those of the indicator displayed first. The control unit 65 may output the message by sound.

Note that a statistical amount indicating the statistical fluctuation may be an instantaneous value of a square sum of the components of the acceleration. In a stationary state, irrespective of the posture of the head 101a, the instantaneous value of the square sum of the components of the acceleration takes a substantially fixed value. When the absolute value of the difference between the calculated square sum and the fixed value is larger than a predetermined value, the control unit 65 determines that the head 101a is not in the stationary state. Instead of using the instantaneous value of the square sum of the components of the acceleration, when a situation in which the absolute value of the difference between the square sum and the fixed value is smaller than the predetermined value continues for a predetermined period, the control unit 65 may determine that the head 101a is stationary.

The reference value set as explained above is a detection value of the sensor 4 corresponding to a posture (hereinafter referred to as reference posture as well) of the head 101a at the time when the user 101 is viewing the center of the screen of the display device 3. In contrast, when the head 101a tilts from the reference posture, the detection value of the sensor 4 changes. In the first embodiment, an operation input corresponding to the posture of the head 101a of the user 101 is created from the difference between the detection value and the reference value of the sensor 4.

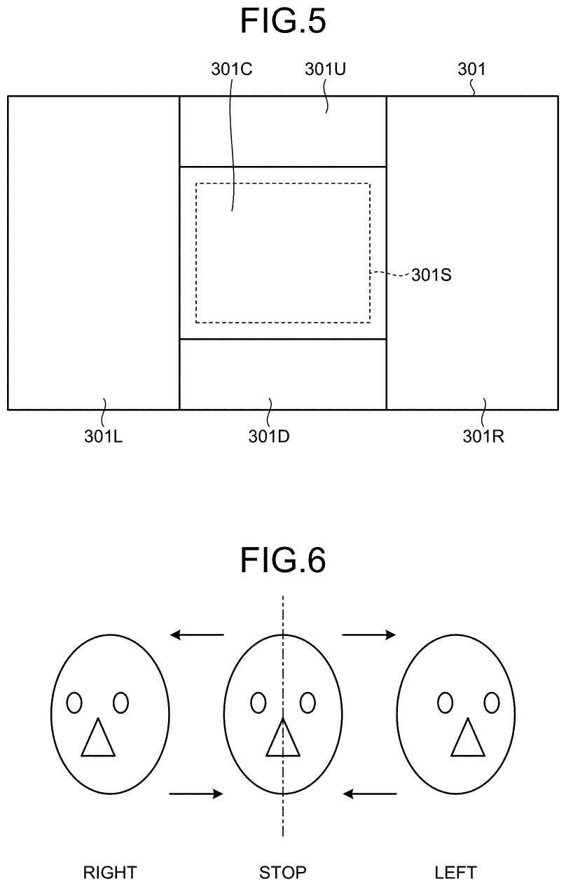

FIG. 5 illustrates an example in which the difference between the detection value of the sensor 4 corresponding to the posture of the head 101a of the user 101 and the reference value is converted into an operation input. The difference between the detection value and the reference value of the sensor 4 is projected onto an operation instruction creation plane 301. The origin of the operation instruction creation plane 301 represents the reference value. The vertical axis of the operation instruction creation plane 301 corresponds to the operation illustrated in FIG. 3 and tilting the head 101a rearward from the reference posture corresponds to moving in the upward direction from the origin of the operation instruction creation plane 301. Similarly, the horizontal axis of the operation instruction creation plane 301 corresponds to the operation illustrated in FIG. 4. Tilting the head 101a to the right from the reference posture corresponds to moving in the right direction from the origin of the operation instruction creation plane 301. In the case illustrated in FIG. 5, the operation instruction creation plane 301 is imaginarily divided into five regions by boundaries indicated by solid lines.

Specifically, the operation instruction creation plane 301 is divided into five rectangular regions, that is, a center region 301C, an upper region 301U, a lower region 301D, a left region 301L, and a right region 301R. The center region 301C is surrounded by the other regions in four directions and includes the center of the operation instruction creation plane 301. The width in the left-right direction of the upper region 301U and the lower region 301D is equal to the width of the center region 301C. The upper region 301U and the lower region 301D are respectively located above and below the center region 301C. The left region 301L and the right region 301R are respectively located on the left and the right of the center region 301C.

After setting the reference value of the sensor 4, the control unit 65 projects the difference between the reference value and the detection value onto the operation instruction creation plane 301. The control unit 65 causes the storing unit 66 to store in which region of the operation instruction creation plane 301 a projected point is. The difference between the reference value and the detection value means a difference between three-dimensional vectors.

For example, when the difference between the detection value and the reference value of the sensor 4 is acceleration corresponding to the upper region 301U and dispersions of components of the acceleration are equal to or smaller than the threshold, the control unit 65 generates an operation instruction signal for moving a visual field upward and transmits the operation instruction signal to the control unit 57 of the microscope device 5. This corresponds to a situation in which the user 101 tilts the head 101a rearward. On the other hand, when any one of the dispersions of the components of the acceleration is larger than the threshold, the control unit 65 does not generate the operation instruction signal.

When the user 101 returns the head 101a to the reference posture from the tilted state, the control unit 65 stops the generation of the operation instruction signal. Specifically, when the detection value of the sensor 4 is acceleration equivalent to the inside of a stop determination region 301S set on the inside of the center region 301C and dispersions of components of the acceleration are equal to or smaller than the threshold, the control unit 65 stops the generation of the operation instruction signal. When the control unit 65 stops the generation of the operation instruction signal halfway in moving the visual field, it is necessary that the posture of the head 101a may be surely determined as being on the inner side of the center region 301C and is the same posture as or a posture close to the reference posture. That is, the absolute value of the difference between the reference value and the detection value at the time when the control unit 65 transitions from a state in which the control unit 65 is generating the operation instruction signal to a state in which the control unit 65 stops the generation of the operation instruction signal is larger than the absolute value of the difference between the reference value and the detection value at the time when the control unit 65 transitions from the state in which the control unit 65 stops the generation of the operation instruction signal to a state in which the control unit 65 starts the generation of the operation instruction signal.

In the first embodiment, by providing the stop determination region 301S on the inside of the center region 301C, for example, when the posture of the head 101a is present near a boundary between the reference posture and the tilted posture, the control unit 65 does not stop the generation of the operation instruction signal. Therefore, the user 101 may not stop operation unless the user 101 brings the head 101a closer to the reference posture. Therefore, it is possible to prevent the visual field movement from stopping against the will of the user 101.

Note that, when the detection value of the sensor 4 greatly deviates from the reference value and is a value with which it may be determined that a place viewed by the user 101 is off the screen, the control unit 65 does not generate the operation instruction signal. When a temporal change of the detection value of the sensor 4 is more sudden than a predetermined reference, the control unit 65 does not generate the operation instruction signal either.

According to the first embodiment explained above, the operation instruction signal for the imaging device is generated using the reference value set by the user viewing the initialization information displayed by the display device and the detection value of the sensor that detects the posture of the head of the user. Therefore, it is possible to operate the imaging device with simple operation.

According to the first embodiment, it is possible to realize visual field movement, zoom and focus driving, and the like of the imaging device with intuitive operation without using a hand of the user.

According to the first embodiment, the acceleration sensor is used as mean for detecting the posture of the head of an operator. Therefore, it is possible to realize a medical observation system having a configuration simpler than the configuration of the technique of Patent Literature 1 described above.

### Modifications

In addition to setting the reference value every time the user 101 wears the eyeglasses 201, the reference value may be set for each posture at the time when the user 101 tilts the head 101a to the front and the rear and the left and the right. In this case, the control unit 65 displays indicators respectively in predetermined positions in the upper region 301U, the lower region 301D, the left region 301L, and the right region 301R on the operation instruction creation plane 301 illustrated in FIG. 5 and urges the user 101 to gaze at the indicators to thereby set the reference value for each posture of the head 101a. Consequently, the reference value corresponding to the posture of the head 101a may be finely set. Note that, for example, the indicators in the left region 301L and the right region 301R may be indicators obtained by rotating a cross to match the top and the bottom and the left and the right at the time when the user 101 views the cross in states in which the user 101 tilts the head 101a on the screen or may be a straight line inclined to match the left and the right at the time when the user 101 views the straight line in the state in which the user 101 tilts the head 101a.

The sensor 4 may further include a three-axis gyro sensor. In this case, the sensor 4 detects rotation of the head 101a illustrated in FIG. 6, the rotation being rotation in the left-right direction (the horizontal direction) in which the height of the eyes of the user 101 is not changed, in other words, rotation passing the center of the head 101a of the user 101 and centering on an axis parallel to the body height direction (the height direction) of the user 101. Consequently, the control unit 65 moves the imaging visual field of the microscope device 5 in the left-right direction based on a detection value of the sensor 4 corresponding to the rotation in the horizontal direction of the head 101a.

In the first embodiment, the input of the operation instruction signal for moving the imaging visual field of the imaging unit 53 of the microscope device 5 according to the posture of the head 101a is received. However, an operation instruction signal for changing the focus and the zoom of the imaging unit 53 according to the posture of the head 101a may be received. For example, postures in which the head 101a tilts in the front-rear direction may be respectively associated with OUT and IN of the focus. On the other hand, postures in which the head 101a tilts in the left-right direction may be respectively associated with OUT and IN of the zoom. An operation mode for moving the imaging visual field according to an operation input to the input unit 55 or the input unit 63 and an operation mode for changing the focus/the zoom according to the operation input may be alternatively selectable.

### Second Embodiment

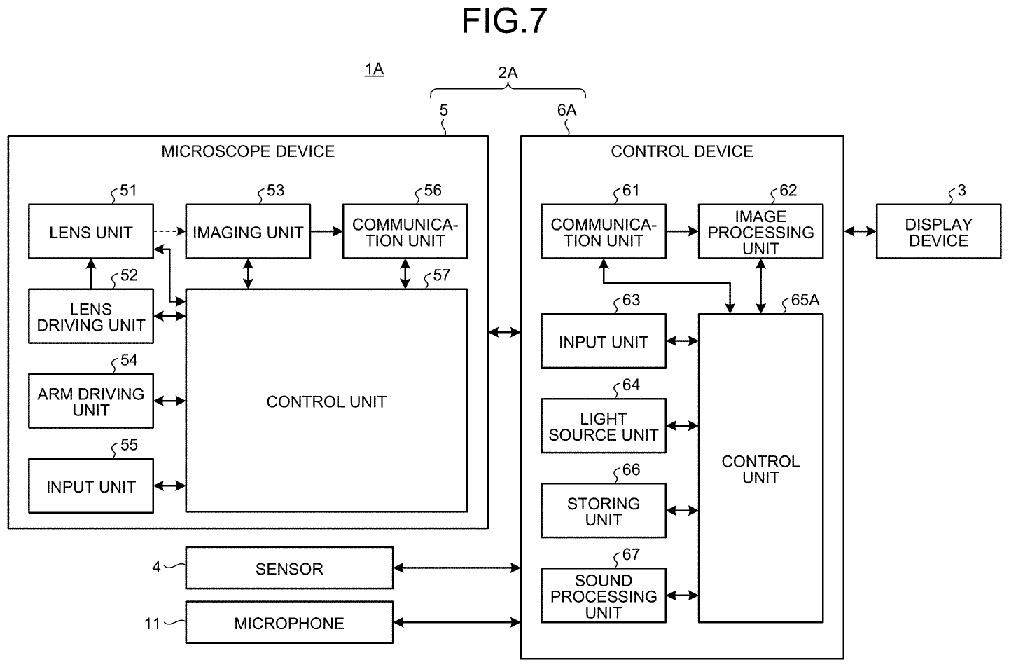

FIG. 7 is a block diagram illustrating the configuration of a medical observation system according to a second embodiment. A medical observation system 1A illustrated in FIG. 7 includes a medical observation apparatus 2A, the display device 3, the sensor 4, and a microphone 11. Note that the same components as the components of the medical observation system 1 explained in the first embodiment are denoted by the same reference numerals and signs and explained.

The microphone 11 is communicatively connected to a control device 6A by radio or wire. The microphone 11 receives a sound input of the user 101 and transmits a sound signal to the control device 6A.

The control device 6A includes the communication unit 61, the image processing unit 62, the input unit 63, the light source unit 64, a control unit 65A, the storing unit 66, and a sound processing unit 67. The sound processing unit 67 executes recognition processing for the sound signal received from the microphone 11. The sound processing unit 67 recognizes content of the sound signal by comparing a feature data of the sound signal and a feature data stored by the storing unit 66.

The medical observation system 1A detects a posture of the head 101a in the case in which the user 101 tilts the head 101a in any one direction of the front and the rear and the left and the right and receives an input of an operation instruction signal corresponding to the detection result. The medical observation system 1A is capable of setting a plurality of operation modes for generating operation instruction signals different from one another. Specifically, the medical observation system 1A is capable of setting an operation mode for any one of an imaging visual field, zoom magnification, and a focus position. A case in which the user 101 sets an operation mode for operating zoom magnification is explained as an example below.

First, the user 101 utters, for example, “wake up” to thereby enable a sound input. When the sound processing unit 67 determines that utterance content of sound acquired from the microphone 11 is “wake up”, the control unit 65A enables a sound input. Note that, when the sound input is enabled, the user 101 may input an instruction signal with the input unit 55 of the microscope device 5 or the input unit 63 of the control device 6A.

Thereafter, the user 101 performs a sound input for selecting a desired operation mode. For example, when selecting the operation mode for operating the zoom magnification, the user 101 utters “zoom”. When the sound processing unit 67 recognizes a sound signal acquired via the microphone 11 as “zoom”, thereafter, the control unit 65A generates an operation instruction signal (zoom-in or zoom-out) concerning zoom according to a detection value of the sensor 4. For example, when coping with a posture in which the head 101a tilts rearward, the control unit 65 generates an operation instruction signal equivalent to the zoom-out. When coping with a posture in which the head 101a tilts forward, the control unit 65 generates an operation instruction signal equivalent to the zoom-in.

When disabling the sound input and the operation by the head 101a, the user utters “cancel”. When the sound processing unit 67 recognizes the sound signal acquired by the microphone 11 as “cancel”, the control unit 65A disables the sound input and the operation by the head 101a and stops the zoom operation.

Note that the expressions such as “wake up”, “zoom”, and “cancel” explained above are only examples. Other expressions may be adopted.

According to the second embodiment explained above, as in the first embodiment, it is possible to operate the imaging device with simple operation.

According to the second embodiment, after selecting the operation mode with sound, the user moves the head and performs the input of the operation instruction signal. Therefore, it is possible to consciously perform selection of the operation mode and prevent wrong operation.

### Third Embodiment

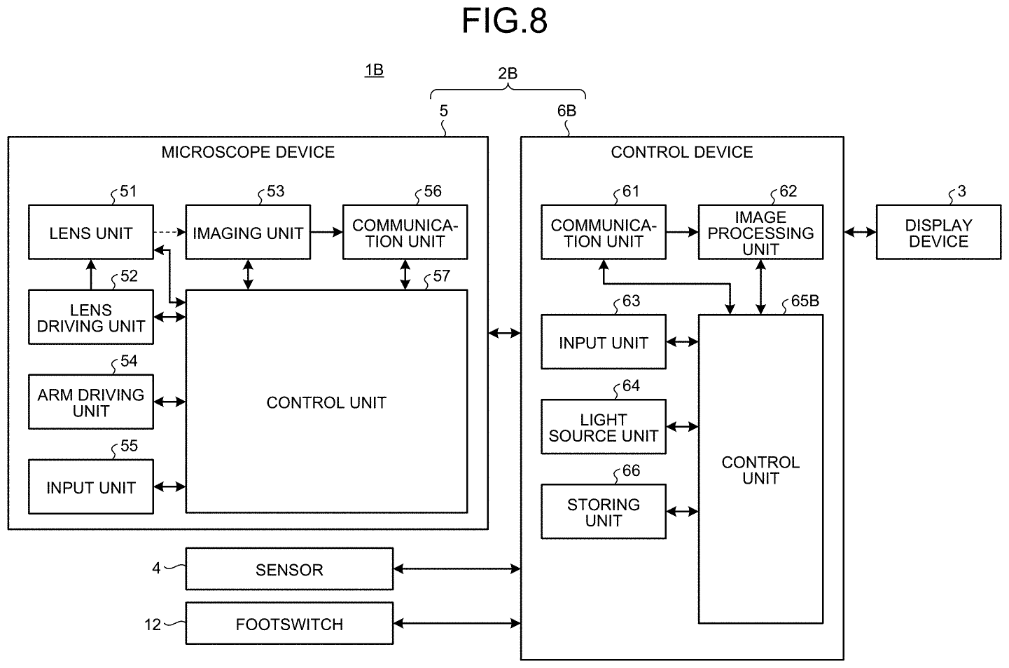

FIG. 8 is a block diagram illustrating the configuration of a medical observation system according to a third embodiment. A medical observation system 1B illustrated in FIG. 8 includes a medical observation apparatus 2B, the display device 3, the sensor 4, and a footswitch 12. Note that the same components as the components of the medical observation system 1 explained in the first embodiment are denoted by the same reference numerals and signs and explained.

Like the medical observation system 1A, the medical observation system 1B has a function with which the user 101 tilts the head 101a in any direction of the front and the rear and the left and the right to thereby selectively execute movement of an imaging visual field, a change of zoom magnification, and a change of a focus position.

The footswitch 12 is communicatively connected to a control device 6B by radio or wire. The footswitch 12 receives an input when the footswitch 12 is stepped in a predetermined amount or more by a foot of the user 101 and transmits an input signal to the control device 6B. The footswitch 12 being stepped in the predetermined amount or more is hereinafter referred to as the footswitch 12 is pressed.

The control device 6B includes the communication unit 61, the image processing unit 62, the input unit 63, the light source unit 64, a control unit 65B, and the storing unit 66. While the footswitch 12 is pressed and an input signal is received, the control unit 65B generates an operation instruction signal corresponding to a posture of the head 101a and transmits the operation instruction signal to the control unit 57 of the microscope device 5.

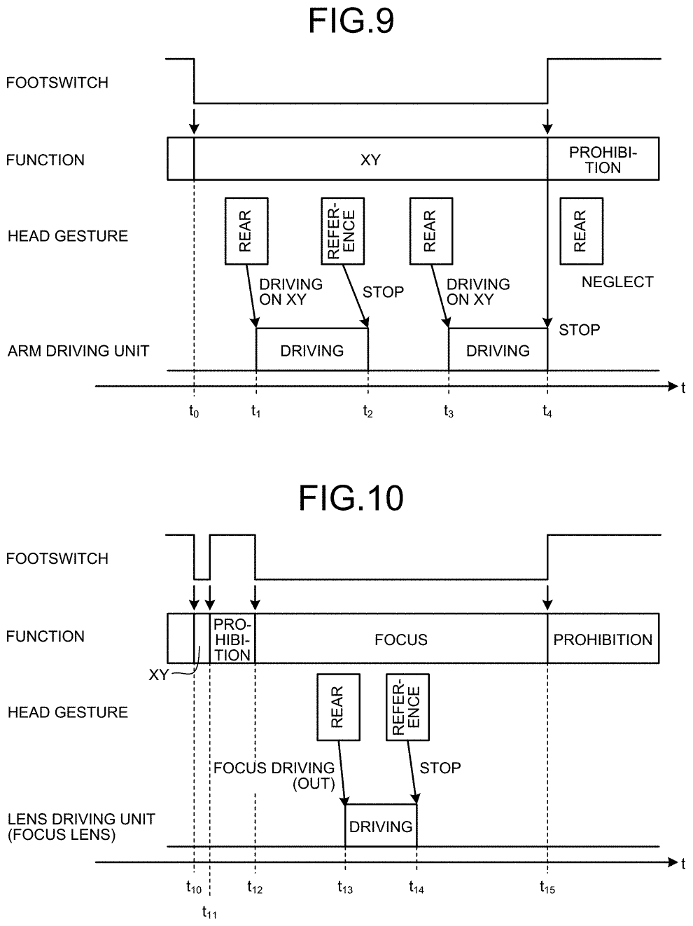

FIGS. 9 to 11 are timing charts illustrating a relation between an input state of the footswitch 12 and the operation of the microscope device 5. First, operation illustrated in FIG. 9 is explained. When the footswitch 12 is pressed at t=t₀, the microscope device 5 changes to an operation mode in which visual field movement (XY) is executable. When the head 101a changes to a posture tilted rearward while the footswitch 12 is kept pressed, the control unit 65B generates an operation instruction signal for moving the imaging visual field upward and transmits the operation instruction signal to the microscope device 5. The arm driving unit 54 starts driving under the control by the control unit 57 (t=t₁). Thereafter, when the head 101a returns to the reference posture, the control unit 65B stops the generation of the operation instruction signal. The arm driving unit 54 stops the operation under the control by the control unit 57 (t=t₂). Subsequently, when the head 101a changes to the posture tilted rearward again, the control unit 65B generates an operation instruction signal for moving the imaging visual field upward and transmits the operation instruction signal to the microscope device 5. The arm driving unit 54 starts driving again under the control by the control unit 57 (t=t₃). Thereafter, when the footswitch 12 is released (t=t₄), the function of the visual field movement is prohibited. Even in a state in which the head 101a is tilted rearward, the control unit 65B stops the generation of the operation instruction signal.

Operation illustrated in FIG. 10 is explained. In FIG. 10, a case in which the operation mode is changed halfway is illustrated. When the footswitch 12 is pressed at t=t₁₀, the microscope device 5 changes to the operation mode in which the visual field movement (XY) is executable. Thereafter, when the pressing of the footswitch 12 is released, the microscope device 5 changes to a visual field movement execution prohibited state (t=t₁₁). When the footswitch 12 is pressed again in an elapsed time from t=t₁₁ shorter than a predetermined time Δt (t=t₁₂<t₁₁+Δt), the control unit 65B changes the operation mode to an operation mode in which a focus function is executable. Thereafter, when the head 101a changes to a posture tilted rearward while the footswitch 12 is kept pressed, the control unit 65B generates an operation instruction signal for the focus-out and transmits the operation instruction signal to the microscope device 5. The lens driving unit 52 starts the focus-out under the control by the control unit 57 (t=t₁₃). Subsequently, when the head 101a returns to the reference posture, the control unit 65B stops the generation of the operation instruction signal. The lens driving unit 52 stops the operation under the control by the control unit 57 (t=t₁₄). Thereafter, when the footswitch 12 is released (t=t₁₅), the function of the focus is prohibited. Note that, when Δt or more elapses thereafter and the footswitch 12 is pressed, the microscope device 5 may be initialized to the operation mode in which the visual field movement is executable or may continue the operation mode in which the focus function is executable.

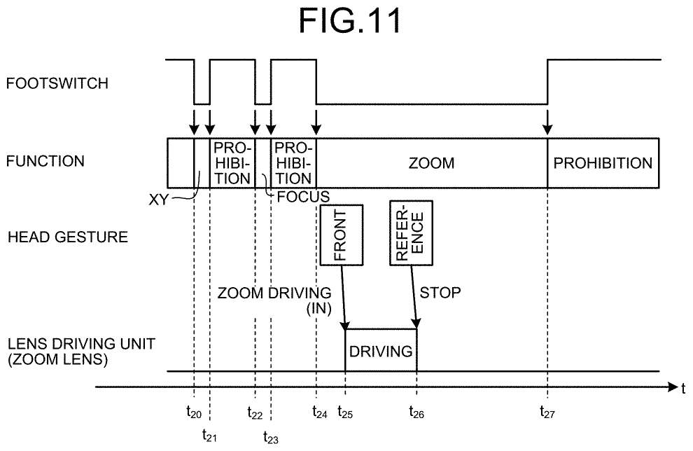

Operation illustrated in FIG. 11 is explained. In FIG. 11, a case in which the operation mode is changed twice halfway is illustrated When the footswitch 12 is pressed at t=t₂₀, the microscope device 5 changes to the operation mode in which the visual field movement (XY) is executable. Thereafter, when the pressing of the footswitch 12 is released, the microscope device 5 changes to a visual field movement execution prohibited state (t=t₂₁). When the footswitch 12 is pressed again in a time shorter than the time Δt from the release (t=t₂₂<t₁₁+Δt), the control unit 65B changes the operation mode to the operation mode in which the focus function is executable. Thereafter, when the pressing of the footswitch 12 is released (t=t₂₃), the microscope device 5 changes to a focus execution prohibited state. When the footswitch 12 is pressed in a time shorter than the time Δt from the release (t=t₂₄<t₂₃+Δt), the control unit 65B changes the operation mode to the operation mode in which the zoom function is executable. When the head 101a changes to a posture tilted forward while the footswitch 12 is kept pressed, the control unit 65B generates an operation instruction signal for the zoom-in and transmits the operation instruction signal to the microscope device 5. The lens driving unit 52 starts the zoom-in under the control by the control unit 57 (t=t₂₅). Subsequently, when the head 101a returns to the reference posture, the control unit 65B stops the generation of the operation instruction signal. The lens driving unit 52 stops the operation under the control by the control unit 57 (t=t₂₆). Thereafter, when the pressing of the footswitch 12 is released (t=t₂₇), the function of the focus is prohibited. Note that, after the pressing of the footswitch 12 is released, when a time equal to or longer than the time Δt elapses and the footswitch 12 is pressed, the microscope device 5 may be initialized to the operation mode in which the visual field movement is executable or may continue the operation mode in which the zoom function is executable.

In the cases illustrated in FIGS. 10 and 11 as well, when the pressing of the footswitch 12 is released in the state in which the head 101a is tilted from the reference posture, execution of a relevant function may be stopped at that point in time.

According to the third embodiment explained above, as in the first embodiment, it is possible to operate the imaging device with simple operation.

According to the third embodiment, operation is effective only when the user moves the head while pressing the footswitch and performs the input of the operation instruction signal. Therefore, it is possible to prevent wrong operation.

### Fourth Embodiment

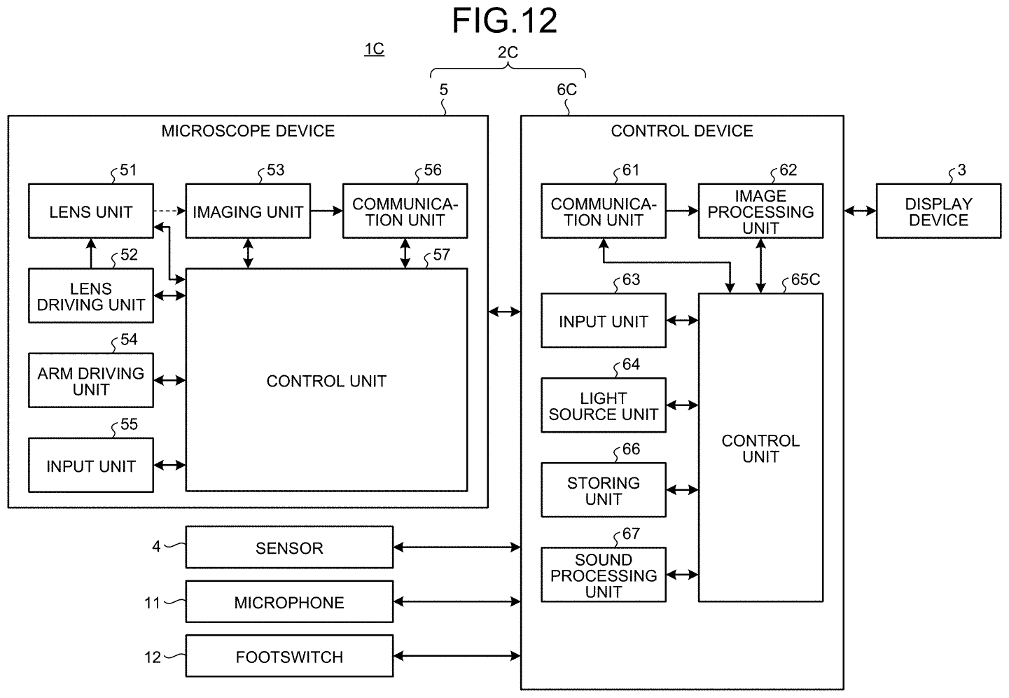

FIG. 12 is a block diagram illustrating the configuration of a medical observation system according to a fourth embodiment. A medical observation system 1C illustrated in FIG. 12 includes a medical observation apparatus 2C, the display device 3, the sensor 4, the microphone 11, and the footswitch 12. Note that the same components as the components of the medical observation system 1 explained in the first embodiment are denoted by the same reference numerals and signs and explained.

Like the medical observation system 1A, the medical observation system 1C has a function with which the user 101 tilts the head 101a in any direction of the front and the rear and the left and the right to thereby selectively execute movement of an imaging visual field, a change of zoom magnification, and a change of a focus position.

A control device 6C includes the communication unit 61, the image processing unit 62, the input unit 63, the light source unit 64, a control unit 65C, the storing unit 66, and the sound processing unit 67. While the footswitch 12 is pressed and an input signal is received, the control unit 65C generates an operation instruction signal corresponding to a sound input via the microphone 11 and a posture of the head 101a and transmits the operation instruction signal to the control unit 57 of the microscope device 5.

After pressing the footswitch 12, the user enables a sound input as in the second embodiment. Subsequent processing corresponding to the posture of the head 101a is the same as the processing in the second embodiment.

However, in the fourth embodiment, the control unit 65C stops the generation of the operation instruction signal corresponding to the sound input and the posture of the head 101a at a point in time when the pressing of the footswitch 12 is released.

According to the fourth embodiment explained above, as in the first embodiment, it is possible to operate the imaging device with simple operation.

According to the fourth embodiment, it is possible to input the operation instruction signal based on the sound input via the microphone and the posture of the head only when the user is consciously pressing the footswitch. Therefore, it is possible to perform operation according to the will of the user and prevent wrong operation.

The modes for carrying out the present disclosure are explained above. However, the present disclosure should not be limited only by the first to fourth embodiments explained above. For example, the imaging device may be an exoscope or an endoscope.

(1) A control device including:

an acquiring unit configured to acquire a detection value from a sensor that detects a posture of a head of a user who operates an imaging device that images an observation target; and

a control unit configured to generate an operation instruction signal for the imaging device using a reference value set by the user viewing initialization information displayed by a display device and the detection value.

(2) The control device according to (1), wherein the control unit is configured to

cause the display device to display the initialization information in a center of a screen of the display device and

associate a display area of the display device and the detection value corresponding to the posture of the head of the user to thereby generate the operation instruction signal.

(3) The control device according to (1) or (2), wherein the control unit is configured to generate the operation instruction signal based on a difference between the reference value and the detection value.
(4) The control device according to (3), wherein an absolute value of the difference at a time when the control unit transitions from a state in which the control unit is generating the operation instruction signal to a state in which the control unit stops the generation of the operation instruction signal is larger than an absolute value of the difference at a time when the control unit transitions from the state in which the control unit stops the generation of the operation instruction signal to a state in which the control unit starts the generation of the operation instruction signal.
(5) The control device according to any one of (1) to (4), wherein the control unit is configured to calculate a statistical amount indicating statistical fluctuation in a predetermined period of the detection value and, disable the operation instruction signal in a case the statistical amount does not satisfy a standard with which it may be determined that the fluctuation is small.
(6) The control device according to any one of (1) to (5), wherein the control unit is configured to disable the operation instruction signal in a case the detection value does not satisfy a standard with which it may be determined that the user is viewing the display device.
(7) The control device according to any one of (1) to (6), wherein the control unit is configured to set a plurality of operation modes for generating the operation instruction signals different from one another.
(8) The control device according to (7), wherein

the acquiring unit is configured to acquire a sound signal of the user, an input of which is received by a microphone,

the control device further includes a sound processing unit configured to recognize content of the sound signal, and

the control unit is configured to change the operation mode according to a recognition result of the sound processing unit.

(9) The control device according to (8), wherein

the acquiring unit is configured to acquire an input signal from a footswitch, and

the control unit is configured to enable an input of the sound signal only in a case where the acquiring unit acquires the sound signal from the microphone while the acquiring unit is acquiring the input signal from the footswitch and generates the operation instruction signal only when the acquiring unit acquires the detection value while the acquiring unit is acquiring the input signal from the footswitch.

(10) The control device according to any one of (1) to (7), wherein

the acquiring unit is configured to acquire an input signal from a footswitch, and

the control unit is configured to generate the operation instruction signal only in a case where the acquiring unit acquires the detection value while the acquiring unit is acquiring the input signal from the foot switch.

(11) The control device according to (10), wherein the control unit is configured to change an operation mode of the imaging device in a case where the acquiring unit acquires the input signal from the footswitch within a predetermined time after the input signal from the footswitch stops.
(12) The control device according to any one of (1) to (11), wherein

the sensor includes an acceleration sensor, and

the sensor is configured to detect a posture in which a head of the user is tilted to any direction of a front and a rear and left and right from a reference posture.

(13) The control device according to (12), wherein the sensor further includes a gyro sensor and is configured to detect rotation from the reference posture of the head of the user, the rotation being rotation in a horizontal direction in which height of eyes of the user is not changed.
(14) The control device according to any one of (1) to (13), wherein

the display device is configured to display a three-dimensional image, and

the sensor is attached to eyeglasses worn by the user in order to view the three-dimensional image.

(15) The control device according to any one of (1) to (14), wherein the operation instruction signal is a signal indicating operation of at least one of visual field movement, zoom, and focus of the imaging device.
(16) A medical observation system including:

an imaging device configured to image a very small part of a body to be observed;

a sensor configured to detect movement of a head of a user who operates the imaging device;

a display device configured to display an image acquired by the imaging device; and

a control device including
an acquiring unit configured to acquire a detection value from the sensor, and
a control unit configured to generate an operation instruction signal for the imaging device using a reference value set by the user viewing initialization information displayed by the display device and the detection value, and output the operation instruction signal to the imaging device.

According to the present disclosure, it is possible to operate the imaging device with simple operation.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A control device comprising:
an acquiring unit configured to acquire a detection value from a sensor that detects a posture of a head of a user who operates an imaging device that images an observation target; and
a control unit configured to generate an operation instruction signal for the imaging device using a reference value set by the user viewing initialization information displayed by a display device and the detection value.

2. The control device according to claim 1, wherein the control unit is configured to
cause the display device to display the initialization information in a center of a screen of the display device and
associate a display area of the display device and the detection value corresponding to the posture of the head of the user to thereby generate the operation instruction signal.

3. The control device according to claim 1, wherein the control unit is configured to generate the operation instruction signal based on a difference between the reference value and the detection value.

4. The control device according to claim 3, wherein an absolute value of the difference at a time when the control unit transitions from a state in which the control unit is generating the operation instruction signal to a state in which the control unit stops the generation of the operation instruction signal is larger than an absolute value of the difference at a time when the control unit transitions from the state in which the control unit stops the generation of the operation instruction signal to a state in which the control unit starts the generation of the operation instruction signal.

5. The control device according to claim 1, wherein the control unit is configured to calculate a statistical amount indicating statistical fluctuation in a predetermined period of the detection value and, disable the operation instruction signal in a case where the statistical amount does not satisfy a standard with which it may be determined that the fluctuation is small.

6. The control device according to claim 1, wherein, the control unit is configured to disable the operation instruction signal in a case where the detection value does not satisfy a standard with which it may be determined that the user is viewing the display device.

7. The control device according to claim 1, wherein the control unit is configured to set a plurality of operation modes for generating the operation instruction signals different from one another.

8. The control device according to claim 7, wherein
the acquiring unit is configured to acquire a sound signal of the user, an input of which is received by a microphone,
the control device further comprises a sound processing unit configured to recognize content of the sound signal, and
the control unit is configured to change the operation mode according to a recognition result of the sound processing unit.

9. The control device according to claim 8, wherein
the acquiring unit is configured to acquire an input signal from a footswitch, and
the control unit is configured to enable an input of the sound signal only when the acquiring unit acquires the sound signal from the microphone while the acquiring unit is acquiring the input signal from the footswitch and generates the operation instruction signal only in a case where the acquiring unit acquires the detection value while the acquiring unit is acquiring the input signal from the footswitch.

10. The control device according to claim 1, wherein
the acquiring unit is configured to acquire an input signal from a footswitch, and
the control unit is configured to generate the operation instruction signal only in a case the acquiring unit acquires the detection value while the acquiring unit is acquiring the input signal from the footswitch.

11. The control device according to claim 10, wherein the control unit is configured to change an operation mode of the imaging device in a case where the acquiring unit acquires the input signal from the footswitch within a predetermined time after the input signal from the footswitch stops.

12. The control device according to claim 1, wherein
the sensor includes an acceleration sensor, and
the sensor is configured to detect a posture in which a head of the user is tilted to any direction of a front and a rear and left and right from a reference posture.

13. The control device according to claim 12, wherein the sensor further includes a gyro sensor and is configured to detect rotation from the reference posture of the head of the user, the rotation being rotation in a horizontal direction in which height of eyes of the user is not changed.

14. The control device according to claim 1, wherein
the display device is configured to display a three-dimensional image, and
the sensor is attached to eyeglasses worn by the user in order to view the three-dimensional image.

15. The control device according to claim 1, wherein the operation instruction signal is a signal indicating operation of at least one of visual field movement, zoom, and focus of the imaging device.

16. A medical observation system comprising:
an imaging device configured to image a very small part of a body to be observed;
a sensor configured to detect movement of a head of a user who operates the imaging device;
a display device configured to display an image acquired by the imaging device; and
a control device including
an acquiring unit configured to acquire a detection value from the sensor, and
a control unit configured to generate an operation instruction signal for the imaging device using a reference value set by the user viewing initialization information displayed by the display device and the detection value, and output the operation instruction signal to the imaging device.
